(19) United States
(12) Patent Application Publication
(10) Pub. No.: US 2008/0319078 A1
(43) Pub. Date: Dec. 25, 2008

(54) Triphenylethylene Compounds Useful as Selective Estrogen Receptor Modulators

(76) Inventor: Subba Reddy Katamreddy, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/093,967

(22) PCT Filed: Nov. 22, 2006
(86) PCT No.: PCT/US2006/045111

§ 371 Date: May 16, 2008

Related U.S. Application Data
(60) Provisional application No. 60/738,772, filed on Nov. 22, 2005.

(51) Int. Cl. A61K 31/192; C07C 63/66; A61P 15/12; A61P 3/10; A61P 35/00

(52) U.S. Cl. 514/571; 562/405

(57) ABSTRACT

Triphenylethylene compounds of formula (I) are provided. The compounds are particularly useful for selective estrogen receptor modulation.

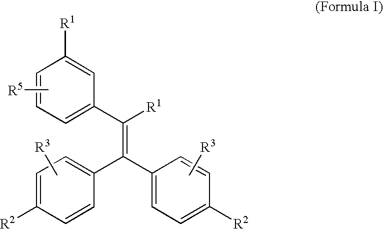

### FIELD OF THE INVENTION

The present invention relates to novel compounds with a variety of therapeutic uses, more particularly to symmetrical triphenyl compounds that are particularly useful for selective estrogen receptor modulation (SERM).

### BACKGROUND OF THE INVENTION

Estrogens are well-known endocrine regulators in the cellular processes involved in the development and maintenance of the reproductive system. Estrogens have also been shown to have important effects in many non-reproductive tissues such as bone, liver, the cardiovascular system, and the central nervous system. The most widely accepted hypothesis of how estrogens exert their effects is by binding to an intracellular steroid hormone receptor. After the receptor and bound ligand are transferred to the nucleus of the cell, the complex binds to recognition sites in DNA, which allows for the modulation of certain genes. Additionally, it is now becoming apparent that estrogens may mediate their effects via membrane-initiated signaling cascade, though much of this work is still experimental Kousteni et al., Journal of Clinical Investigation, (2003), 111, 1651-1664, herein incorporated by reference with regard to such teaching.

Certain substances have demonstrated the ability to exhibit their biological activity in a “issue-selective” manner. In other words, tissue selectivity allows functionality as estrogen agonists in certain tissues, while acting as estrogen antagonists in other tissues. The term “selective estrogen receptor modulators” (SERMs) has been given to these molecules. Examples of SERMs include tamoxifen, raloxifene, lasofoxifene, clomiphene, and nafoxidine. The molecular basis for this tissue-selective activity is not completely understood. Without being limited to any particular theory, the ability of the ligand to place the estrogen receptor into different conformational states and allowing for differential capabilities in recruiting coactivator and corepressor proteins, as well as other important proteins involved in transcriptional regulation, is believed to play a role. See, McDonnell, D P., The Molecular Pharmacology of SERMs, Trends Endocrinol. Metab. 1999, 301-311, herein incorporated by reference with regard to such description.

Historically estrogens were believed to manifest their biological activity through a single estrogen receptor, now termed estrogen receptor alpha (ERα). More recently, however, there was the discovery of second subtype of estrogen receptor, termed estrogen receptor beta (ERβ). See, Kuiper et al., WO 97/09348 and Kuiper et al., Cloning of a Novel Estrogen Receptor Expressed in Rat Prostate and Ovary, Proc. Natl. Acad. Sci. U.S.A., 1996, pp. 5925-5930, herein incorporated by reference with regard to such subtype. ERβ is expressed in humans. See, Mosselman et al., ERβ: Identification and Characterization of a Novel Human Estrogen Receptor, FEBR S Lett., 1996, pp. 49-53, herein incorporated by reference with regard to such expression. The discovery of this second subtype of estrogen receptor significantly increased the biological complexity of estrogen signaling and may be responsible for some of the tissue-selective actions of the currently available SERMs.

As noted above, estrogens have important effects in many non-reproductive tissues. Thus, estrogen modulation is believed useful in the treatment and/or prophylaxis of diseases and conditions associated with such tissues, including bone, liver, and the central nervous system. For example, osteoporosis is characterized by the net loss of bone mass per unit volume. Such bone loss results in a failure of the skeleton to provide adequate structural support for the body, thereby creating an increased risk of fracture. One of the most common types of osteoporosis is postmenopausal osteoporosis, which is associated with accelerated bone loss subsequent to cessation of menses and declining levels of endogenous estrogen in women. There is an inverse relationship between densitometric measures of bone mass and fracture risk, for peri- and postmenopausal women in the process of rapid bone loss due to declining levels of estrogen. See, Slemenda, et al., Predictors of Bone Mass in Perimenopausal Women, A Prospective Study of Clinical Data Using Photon Abr sorptiometry, Ann. Intern. Med., 1990, pp. 96-101 and Marshall, et al., Meta-Analysis of How Well Measures of Bone Mineral Density Predict Occurrence of Osteoporotic Fractures, Br Med. J., 1996, pp. 1254-1259, each of which is herein incorporated by reference with regard to such relationship. Elderly women currently have a lifetime risk of fractures of about 75%. In addition there is an approximate 40% risk of hip fracture for Caucasian women over age 50 in the United States. The economic burden from osteoporotic fractures is considerable because of the necessity of hospitalization. In addition, although osteoporosis is generally not thought of as life-threatening, the mortality within 4 months of hip fracture is currently approximately 20 to 30%. Current therapies for postmenopausal osteoporosis include hormone replacement therapy or treatment with other antiresorptive agents such as bisphosphonates or calcitonin. Similarly, SERMS have been shown to be effective in the treatment of postmenopausal osteoporosis (see, Lindsay, R.: Sex steroids in the pathogenesis and prevention of osteoporosis. In: Osteoporosis 1988. Etiology, Diagnosis and Management. Riggs B L (ed)I, Raven Press, New York, USA (1988):333-358; Barzel US: Estrogens in the prevention and treatment of postmenopausal osteoporosis: a review. Am J. Med (1988) 85:847-850; and Ettinger, B., Black, D. M., et al., Reduction of Vertebral Fracture Risk in Postmenopausal Women with Osteoporosis Treated with Raloxifene, JAMA, 1999, 282, 637-645, each of which is incorporated by reference with regard to such teaching).

As another example, the effects of estrogens on breast tissue, particularly breast cancer, have been well documented. For example, a previously identified SERM, tamoxifen, decreases the risk of recurrent breast cancer, contralateral breast cancer, and mortality as well as increases the disease-free survival rate of patients with breast cancer at multiple stages of the disease. See, Cosman, F., Lindsay, R. Selective Estrogen Receptor Modulators: Clinical Spectrum, Endocrine Rev., 1999, pp. 418-434, herein incorporated by reference with regard to such teaching. The profile of tamoxifen, however, is not ideal due to potential interactive properties on reproductive tissues, such as uterine tissues. There is room for an improved therapy for the treatment of such cancers, namely a SERM with reduced agonist properties on reproductive tissues.

Cardiovascular disease is the leading cause of death among postmenopausal women. Until recently, the preponderance of data suggested that estrogen replacement therapy in postmenopausal women reduced the risk of cardiovascular disease, although some studies reported no beneficial effect on overall mortality. See, Barrett-Connor, E. et al., The Potential of SERMs for Reducing the Risk of Coronary Heart Disease, Trends Endocrinol. Metab., 1999, pp. 320-325, herein incorporated by reference. The mechanism(s) by which estrogens were believed to exert their beneficial effects on the cardiovascular system are not entirely clear. Potentially estrogen's effects on serum cholesterol and lipoproteins, antioxidant properties, vascular smooth muscle proliferation, and inhibition of arterial cholesterol accumulation were believed to play a role. Id. See also, Cosman, F., Lindsay, R. Selective Estrogen Receptor Modulators: Clinical Spectrum, Endocrine Rev., 1999, pp. 418-434, herein incorporated by reference. In light of the recent reports of the HERS II and WHI studies, however, continuous combined Hormone Therapy, namely, CEE+MPA [Conjugated Equine Estrogen+Medroxy Progesterone Acetate], confers no cardiovascular benefit in menopausal women. See, Hulley S., Grady, D., Bush, T., et al., Randomized trial of estrogen plus progestin for secondary prevention of coronary heart disease in postmenopausal women. Heart and Estrogen/progestin Replacement Study (HERS) Research Group. J. Am. Med. Assoc. (1998) 280:605-613 and Wassertheil-Smoller S., Hendrix, S. L., Limacher, M., et al., for the WHI Investigators. Effect of estrogen plus progestin on stroke in postmenopausal women: the Women's Health Initiative: a randomized trial JAMA (2003) 289, 2673-2684, each herein incorporated by reference with regard to such teaching). To what extent these findings may be extrapolated to SERMs is an issue that remains to be determined.

Other therapeutic alternatives include estrogen replacement therapy and/or hormone replacement therapy, which may be useful in the treatment of vasomotor symptoms, genitourinary atrophy, depression, and diabetes. Over 75% of women experience vasomotor symptoms during the climacteric years. Clinical signs, such as vasomotor symptoms and genitourinary atrophy, abate upon treatment with estrogen replacement therapy. Sagraves, R., J. Clin. Pharmacol. (1995), 35(9 Suppl):2S-10S, herein incorporated by reference with regard to such teaching. Preliminary data suggest that estradiol may alleviate depression during perimenopause and that the combination of estrogens and selective serotonin reuptake inhibitors may alleviate depression during the postmenopausal period. Soares, C. N., Poitras, J. R., and Prouty, J., Drugs Aging, (2003), 20(2), 85-100, herein incorporated by reference with regard to such teaching. Furthermore, hormone replacement therapy may improve glycemic control among women with diabetes. Palin, S. L. et al., Diabetes Research and Clinical Practice, (2001), 54, 67-77; Ferrara, A. et al., Diabetes Care, (2001), 24(7), 1144-1150), each incorporated herein by reference with regard to such teaching. There is a need, however, for improved therapies that present better side effect profiles.

### SUMMARY OF THE INVENTION

The present inventors discovered a novel group of symmetrical triphenyl compounds, which bind to and modulate estrogen receptor alpha and estrogen receptor beta. As SERMS, these compounds are believed to be useful for the treatment and/or prophylaxis of conditions such as menopausal or postmenopausal disorders, vasomotor symptoms, urogenital or vulvar vaginal atrophy, atrophic vaginitis, female sexual dysfunction, breast cancer, depressive symptoms, diabetes, bone demineralization, and the treatment and/or prevention of osteoporosis.

The present invention provides a compound of Formula I,

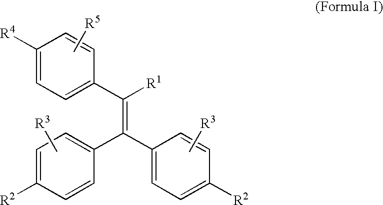

or a pharmaceutically acceptable salt or solvate thereof, wherein
each R³ is the same and selected from the group consisting of hydrogen, hydroxy, C₁-C₆ alkyl, halogen, C₁-C₆ alkoxy, and C₁-C₆ haloalkyl;

### R⁴ is —OCH₂C(O)OH; and

R¹ is selected from the group consisting of C₁-C₆ alkyl and C₁-C₆ haloalkyl;
each R² is the same and selected from the group consisting of hydroxy, C₁-C₄ alkoxy, and halogen; and
R⁵ is selected from the group consisting of hydroxy, C₁-C₆ alkyl, halogen, C₁-C₆ alkoxy, or C₁-C₆ haloalkyl;
   or
R¹ is selected from the group consisting of C₁-C₆ alkyl and C₁-C₆ haloalkyl;
each R² is the same and selected from the group consisting of C₁-C₄ alkoxy, and halogen; and
R⁵ is selected from the group consisting of hydrogen, hydroxy, C₁-C₆ alkyl, halogen, C₁-C₆ alkoxy, or C₁-C₆ haloalkyl;
   or
R¹ is selected from the group consisting of C₃-C₆ alkyl and C₁-C₆ haloalkyl;
each R² is the same and selected from the group consisting of hydroxy, C₁-C₄ alkoxy, and halogen; and
R⁵ is selected from the group consisting of hydrogen, hydroxy, C₁-C₆ alkyl, halogen, C₁-C₆ alkoxy, or C₁-C₆ haloalkyl.

According to an embodiment, a compound of formula I is provided as described in any one of the examples.

According to another embodiment, the invention provides a compound of Formula I, a salt, or a solvate, thereof for use as an active therapeutic substance.

According to another embodiment, the invention provides a pharmaceutical composition comprising compound of Formula I, a salt, or a solvate thereof and a pharmaceutically acceptable carrier.

According to another embodiment, the invention provides a compound of Formula I, a salt, or a solvate thereof for use in the treatment, including phophylaxis, of a condition or disorder affected by selective estrogen receptor modulation.

According to another embodiment, the invention provides the use of a compound of Formula I, or a salt, or a solvate thereof for use in the treatment, which may include phophylaxis, of a condition or disorder affected by selective estrogen receptor modulation.

According to another embodiment, the invention provides the use of a compound of formula I, or a salt, or a solvate thereof in the manufacture of a medicament for use in the treatment, which is used hereinafter to include prophylaxis, of a condition or disorder affected by selective estrogen receptor modulation.

According to another embodiment, the invention provides a method of treatment, which may include phophylaxis, of a condition or disorder affected by selective estrogen receptor modulation in a mammal in need thereof, with a compound of Formula I, or a salt, or a solvate thereof.

According to another embodiment, the present invention provides a method for treating conditions such as those selected from menopausal or postmenopausal disorders, vasomotor symptoms, urogenital or vulvar vaginal atrophy, atrophic vaginitis, endometriosis, female sexual dysfunction, breast cancer, depressive symptoms, diabetes, bone demineralization, and osteoporosis.

### DETAILED DESCRIPTION OF THE INVENTION

The invention herein is described in terms known and appreciated by those skilled in the art. For ease of reference certain terms are defined. The fact that certain terms are defined, however, should not be considered as indicative that any term that is undefined is indefinite. Rather, all terms used are believed to describe the invention in terms such that one of ordinary skill can appreciate the scope and practice the present invention.

As used herein the term “alkyl” refers to a straight or branched chain hydrocarbon having from one to twelve carbon atoms. Examples of “alkyl” as used herein include, but are not limited to, methyl, ethyl, propyl, isopropyl, isobutyl, n-butyl, tert-butyl, isopentyl, n-pentyl, and the like.

As used herein, the term “alkylene” refers to a straight or branched chain divalent hydrocarbon radical having from one to ten carbon atoms. Examples of “alkylene” as used herein include, but are not limited to, methylene, ethylene, n-propylene, n-butylene, and the like.

As used herein the term “halogen” refers to fluorine, chlorine, bromine, or iodine.

As used herein the term “haloalkyl” refers to an alkyl group, as defined herein, which is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups (i.e., trifluoromethyl) and the like.

As used herein the term “alkoxy” refers to the group —OR, where R is alkyl as defined above.

As used herein the term “acyl” refers to the group —C(O)R, where R is alkyl, aryl, heteroaryl, or heterocyclyl, as each is defined herein.

As used herein the term “hydroxy” refers to the group —OH.

As used herein the term “carboxy” refers to the group —C(O)OH.

As used herein the term “nitro” refers to the group —NO₂.

As used herein the term “amino” refers to the group —NH₂, or when referred to as substituted amino defines such groups substituted with alkyl.

As used herein, the term “cycloalkyl” refers to a non-aromatic, saturated or unsaturated, mono- or bi-cyclic hydrocarbon ring having from three to ten carbon atoms. Exemplary “cycloalkyl” groups include, but are not limited to, cyclopropyl, cyclobutyl, cyclopentyl, cyclohexyl, and cycloheptyl.

As used herein, the term “aryl” refers to a phenyl ring or to a phenyl ring system fused to one or more additional phenyl rings to form, for example, anthracene, phenanthrene, or naphthalene ring systems. Examples of “aryl” groups include, but are not limited to, phenyl, 2-naphthyl, 1-naphthyl, biphenyl, and the like.

As used herein, the term “heteroaryl” refers to a monocyclic five to seven membered aromatic ring, or fused bicyclic aromatic ring system comprising two of such monocyclic five to seven membered aromatic rings. These heteroaryl rings contain one to four heteroatoms selected from N, O, and S, where N-oxides, sulfur oxides, and dioxides are permissible heteroatom substitutions. Examples of “heteroaryl” groups used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, and the like.

As used herein, the term “heterocycle” or “heterocyclyl” refers to a non-aromatic, mono- or bi-cyclic ring system containing optionally one or more degrees of unsaturation and also containing one to four heteroatoms selected from N, O and/or S., “Heterocycle” and “heterocyclyl” also includes variants thereof wherein the heteroatom, N or S is substituted by oxo to provide N-oxides and sulfur oxide. Preferred heteroatoms include N, O, or both. Preferably the ring is three to ten-membered and is either saturated or has one or more degrees of unsaturation. Such rings may be optionally fused to one or more of another “heterocyclic” ring(s), heteroaryl ring(s), aryl ring(s), or cycloalkyl ring(s). Examples of “heterocyclic” groups include, but are not limited to, tetrahydrofuran, pyran, 1,4-dioxane, 1,3-dioxane, piperidine, pyrrolidine, morpholine, tetrahydrothiopyran, and tetrahydrothiophene.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

As used herein, the term “solvate” refers to a complex of variable stoichiometry formed by a solute (in this invention, a compound of Formula I, or a salt or physiologically functional derivative thereof) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. Most preferably the solvent used is water.

As used herein, a substituent may be indicated as attached to a ring structure using the following representation:

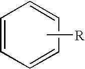

This representation indicates that the R substituent may be located at any point on the ring structure that is not otherwise occupied by specifically specified substituents or groups.

The present invention provides a compound of Formula I:

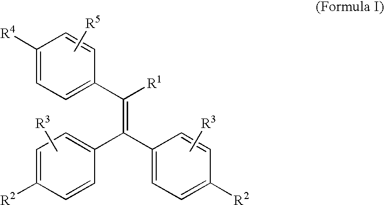

or a pharmaceutically acceptable salt or solvate thereof, wherein
each R³ is the same and selected from the group consisting of hydrogen, hydroxy, C₁-C₆ alkyl, halogen, C₁-C₆ alkoxy, and C₁-C₆ haloalkyl;

### R⁴ is —OCH₂C(O)OH; and

R¹ is selected from the group consisting of C₁-C₆ alkyl and C₁-C₆ haloalkyl;
each R² is the same and selected from the group consisting of hydroxy, C₁-C₄ alkoxy, and halogen; and
R⁵ is selected from the group consisting of hydroxy, C₁-C₆ alkyl, halogen, C₁-C₆ alkoxy, or C₁-C₆ haloalkyl;
   or
R¹ is selected from the group consisting of C₁-C₆ alkyl and C₁-C₆ haloalkyl;
each R² is the same and selected from the group consisting of C₁-C₄ alkoxy, and halogen; and
R⁵ is selected from the group consisting of hydrogen, hydroxy, C₁-C₆ alkyl, halogen, C₁-C₆ alkoxy, or C₁-C₆ haloalkyl;
   or

R¹ is selected from the group consisting of C₃-C₈ alkyl and C₁-C₆ haloalkyl;
each R² is the same and selected from the group consisting of hydroxy, C₁-C₄ alkoxy, and halogen; and
R⁵ is selected from the group consisting of hydrogen, hydroxy, C₁-C₆ alkyl, halogen, C₁-C₆ alkoxy, or C₁-C₆ haloalkyl.

According to a first embodiment of the invention, R¹ is selected from the group consisting of C₁-C₆ alkyl and C₁-C₆ haloalkyl; each R² is the same and selected from the group consisting of hydroxy, C₁-C₄ alkoxy, and halogen; and

R⁵ is selected from the group consisting of hydroxy, C₁-C₆ alkyl, halogen, C₁-C₆ alkoxy, or C₁-C₆ haloalkyl. Advantageously, R¹ of the first embodiment is selected from C₂-C₆ alkyl. Advantageously, R² of the first embodiment is hydroxy.

According to a second embodiment of the invention, R¹ is selected from the group consisting of C₁-C₆ alkyl and C₁-C₆ haloalkyl; each R² is the same and selected from the group consisting of C₁-C₄ alkoxy, and halogen; and R⁵ is selected from the group consisting of hydrogen, hydroxy, C₁-C₆ alkyl, halogen, C₁-C₆ alkoxy, or C₁-C₆ haloalkyl. Advantageously, R¹ of the second embodiment is selected from C₂-C₆ alkyl. Advantageously, R⁵ of the second embodiment is hydrogen.

According to a third embodiment of the invention, R¹ is selected from the group consisting of C₃-C₆ alkyl and C₁-C₆ haloalkyl; each R² is the same and selected from the group consisting of hydroxy, C₁-C₄ alkoxy, and halogen; and R⁵ is selected from the group consisting of hydrogen, hydroxy, C₁-C₆ alkyl, halogen, C₁-C₆ alkoxy, or C₁-C₆ haloalkyl. Advantageously, R¹ of the third embodiment is selected from C₃-C₆ alkyl. Advantageously, R² of the third embodiment is hydroxy. Advantageously, R⁵ of the third embodiment is hydrogen.

Particularly preferred compounds of the present invention include:
({4-[1-butyl-2,2-bis(4-hydroxyphenyl)ethenyl]phenyl}oxy)acetic acid;
({4-[2,2-bis(4-hydroxyphenyl)-1-propylethenyl]phenyl}oxy)acetic acid;
[(4-{1-ethyl-2,2-bis[4-(methyloxy)phenyl]ethenyl}phenyl)oxy]acetic acid; and
{[4-[1-ethyl-2,2-bis(4-hydroxyphenyl)ethenyl]-2-(methyloxy)phenyl]oxy}acetic acid.

The compounds of formulas (I) may crystallize in more than one form, a characteristic known as polymorphism, and such polymorphic forms (“polymorphs”) are within the scope of formula (I). Polymorphism generally can occur as a response to changes in temperature, pressure, or both. Polymorphism can also result from variations in the crystallization process. Polymorphs can be distinguished by various physical characteristics known in the art such as x-ray powder diffraction patterns, infra-red spectra, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by formula (I), as well as any wholly or partially equilibrated mixtures thereof. The present invention also includes the individual isomers of the compounds represented by the formulas above as mixtures with isomers thereof in which one or more chiral centers are inverted.

According to another embodiment, at each occurrence, each alkyl, alkoxy, haloalkyl, and alkylene may be optionally substituted. As used herein throughout the present specification, the phrase “optionally substituted” or variations thereof denote an optional substitution, including multiple degrees of substitution, with one or more substituent group. The phrase should not be interpreted so as to be imprecise or duplicative of substitution patterns herein described or depicted specifically. Rather, those of ordinary skill in the art will appreciate that the phrase is included to provide for obvious modifications, which are encompassed within the scope of the appended claims.

The present invention includes one or more of the compounds of Formula I for use in the treatment of conditions or disorders affected by selective estrogen receptor modulation in a mammal (such as a human) in need thereof. In one embodiment, the present invention provides methods for the treatment of conditions or disorders selected from List A:

List A (conditions or disorders affected by selective estrogen receptor modulation and treatable by the compounds of Formula I): osteoporosis, bone demineralization, reduced bone mass, density, or growth, osteoarthritis, acceleration of bone fracture repair and healing, acceleration of healing in joint replacement, periodontal disease, acceleration of tooth repair or growth, Paget's disease, osteochondrodysplasias, muscle wasting, the maintenance and enhancement of muscle strength and function, frailty or age-related functional decline (“ARFD”), sarcopenia, chronic fatigue syndrome, chronic myaligia, acute fatigue syndrome, acceleration of wound healing, maintenance of sensory function, chronic liver disease, AIDS, weightlessness, burn and trauma recovery, thrombocytopenia, short bowel syndrome, irritable bowel syndrome, inflammatory bowel disease, Crohn's disease and ulcerative colitis, obesity, eating disorders including anorexia associated with cachexia or aging, hypercortisolism and Cushing's syndrome, cardiovascular disease or cardiac dysfunction, congestive heart failure, high blood pressure, breast cancer, malignant tumore cells containing the androgen receptor including breast, brain, skin, ovary, bladder, lymphatic, liver, kidney, uterine, pancreas, endometrium, lung, colon, and prostate, prostatic hyperplasia, hirsutism, acne, seborrhea, androgenic alopecia, anemia, hyperpilosity, adenomas and neoplasis of the prostate, hyperinsulinemia, insulin resistance, diabetes, syndrome X, dyslipidemia, urinary incontinence, artherosclerosis, libido enhancement, sexual dysfunction, depression, depressive symptoms, nervousness, irritability, stress, reduced mental energy and low self-esteem, improvement of cognitive function, endometriosis, polycystic ovary syndrome, counteracting preeclampsia, premenstral syndrome, contraception, uterine fibroid disease, and/or aortic smooth muscle cell proliferation, vaginal dryness, pruritis, dyspareunia, dysuria, frequent urination, urinary tract infections, hypercholesterolemia, hyperlipidemia, peripheral vascular disease, restenosis, vasospasm, vascular wall damage due to immune responses, Alzheimer's disease, bone disease, aging, inflammation, rheumatoid arthritis, respiratory disease, emphysema, reperfusion injury, viral hepatitis, tuberculosis, psoriasis, systemic lupus erythematosus, amyotrophic lateral sclerosis, stroke, CNS trauma, dementia, neurodegeneration, breast pain and dysmenorrhea, menopausal or postmenopausal disorders, vasomotor symptoms, urogenital or vulvar vaginal atrophy, atrophic vaginitis, female sexual dysfunction, for enhancing libido, for the treatment of hypoactive sexual disorder, sexual arousal disorder, for increasing the frequency and intensity of orgasms, vaginismus, osteopenia, endometriosis, BPH (benign prostatic hypertrophy), dysmenorrhea, autoimmune diseases, Hashimoto's thyroiditis, SLE (systemic lupus erythematosus), myasthenia gravis, or reperfusion damage of ischemic myocardium. More preferably the treatment relates to menopausal or postmenopausal disorders, -vasomotor symptoms, urogenital or vulvar vaginal atrophy, atrophic vaginitis, endometriosis, female sexual dysfunction, breast cancer, depressive symptoms, diabetes, bone demineralization, or osteoporosis.

Also, the present invention includes the use of one or more of the compounds of Formula I in the manufacture of a medicament for use in the treatment of conditions or disorders associated with selective estrogen receptor modulation. Preferably the medicament is for use in the treatment of those conditions and disorders of List A, above.

The present invention includes a method for the treatment of conditions or disorders associated with selective estrogen receptor modulation comprising the administration of at least one compound of Formula I. Preferably the treatment relates to the conditions and disorders of List A, above.

The compounds of formula I, or salts or solvates thereof, may be advantageous in the treatment of menopausal or postmenopausal disorders.

The compounds of formula I, or salts or solvates thereof, may be advantageous in the treatment of vasomotor symptoms.

The compounds of formula I, or salts or solvates thereof, may be advantageous in the treatment of urogenital or vulvar vaginal atrophy.

The compounds of formula I, or salts or solvates thereof, may be advantageous in the treatment of atrophic vaginitis.

The compounds of formula I, or salts or solvates thereof, may be advantageous in the treatment of endometriosis.

The compounds of formula I, or salts or solvates thereof, may be advantageous in the treatment of female sexual dysfunction.

The compounds of formula I, or salts or solvates thereof, may be advantageous in the treatment of breast cancer.

The compounds of formula I, or salts or solvates thereof, may be advantageous in the treatment of depressive symptoms.

The compounds of formula I, or salts or solvates thereof, may be advantageous in the treatment of diabetes.

The compounds of formula I, or salts or solvates thereof, may be advantageous in the treatment of bone demineralization.

The compounds of formula I, or salts or solvates thereof, may be advantageous in the treatment of osteoporosis.

In particular, the compounds of the present invention are believed useful, either alone or in combination with other agents, in the treatment of menopausal or postmenopausal disorders, vasomotor symptoms, urogenital or vulvar vaginal atrophy, atrophic vaginitis, female sexual dysfunction, breast cancer, depressive symptoms, diabetes, bone demineralization, and the treatment of osteoporosis.

As used herein, the term “effective amount” means that amount of a drug or pharmaceutical agent that will elicit the biological or medical response of a tissue, system, animal or human that is being sought, for instance, by a researcher or clinician. The term “therapeutically effective amount” means any amount which, as compared to a corresponding subject who has not received such amount, results in treatment, healing, prevention, or amelioration of a disease, disorder, or side effect, or a decrease in the rate of advancement of a disease or disorder. The term also includes within its scope amounts effective to enhance normal physiological function.

A therapeutically effective amount of a compound of the present invention will depend upon a number of factors. For example, the age and weight of the animal, the precise condition requiring treatment and its severity, the nature of the formulation, and the route of administration are all factors to be considered. The therapeutically effective amount ultimately should be at the discretion of the attendant physician or veterinarian. For example, an effective amount of a compound of formula I for the treatment of humans suffering from osteoporosis, generally, should be in the range of 0.1 to 100 mg/kg body weight of recipient (mammal) per day. More usually the effective amount should begin the range of 1 to 10 mg/kg body weight per day. Thus, for a 70 kg adult mammal the actual amount per day would usually be from 70 to 700 mg. This amount may be given in a single dose per day or in a number (such as two, three, four, five, or more) of sub-doses per day such that the total daily dose is the same. An effective amount of a salt or solvate thereof, may be determined as a proportion of the effective amount of the compound of formula I per se. Similar dosages should be appropriate for treatment of the other conditions referred to herein that are mediated by estrogen.

For use in therapy, therapeutically effective amounts of a compound of formula I, as well as salts and solvates thereof, may be administered as the raw chemical. Additionally, the active ingredient may be presented as a pharmaceutical composition. Accordingly, the invention further provides pharmaceutical compositions that include effective amounts of compounds of the Formula I and salts and solvates thereof, and one or more pharmaceutically acceptable carriers, diluents, or excipients. The compounds of Formula I and salts or solvates thereof, are as described above. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the Formula I or salts and solvates thereof, with one or more pharmaceutically acceptable carriers, diluents or excipients.

Pharmaceutical formulations may be presented in unit dose forms containing a predetermined amount of active ingredient per unit dose. Such a unit may contain, as a non-limiting example, 0.5 mg to 1 g of a compound of the formula I, depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like. Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders, such as carboxymethylcellulose, aliginates, gelatins, or polyvinyl pyrrolidone, solution retardants, such as paraffin, resorption accelerators such as a quaternary salt and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of Formula I and salts and solvates thereof, can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of Formula I and salts or solvates thereof may also be delivered by the use of monoclonal antibodies as individual carriers to which the compound molecules are coupled. The compounds may also be coupled with soluble polymers as targetable drug carriers. Such polymers can include polyvinylpyrrolidone (PVP), pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical formulations adapted for topical administrations to the eye include eye drops wherein the active ingredient is dissolved or suspended in a suitable carrier, especially an aqueous solvent. Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring agents.

The compounds of the present invention and their salts or solvates thereof, may be employed alone or in combination with other therapeutic agents for the treatment of the conditions described in List A above. For example, in osteoporosis therapy, combination with other osteoporosis therapeutic agents is envisaged. Osteoporosis combination therapies according to the present invention thus comprise the administration of at least one compound of formula I or a salt or solvate thereof, and the use of at least one other osteoporosis treatment method. Preferably, combination therapies according to the present invention comprise the administration of at least one compound of Formula I or a salt or solvate thereof, and at least one other osteoporosis treatment agent, for example, a bone building agent. As a further example, combination therapies according to the present invention include the administration of at least one compound of the present invention or a salt or solvate thereof, and at least one other osteoporosis treatment agent, for example, an anti-bone resorption agent. As noted, one potential additional osteoporosis treatment agent is a bone building (anabolic) agent. Bone building agents can lead to increases in parameters such as bone mineral density that are greater than those than can be achieved with anti-resorptive agents. In some cases, such anabolic agents can increase trabecular connectivity leading to greater structural integrity of the bone.

The compound(s) of Formula I and the other pharmaceutically active agent(s) may be administered together or separately and, when administered separately, administration may occur simultaneously or sequentially in any order. The amounts of the compound(s) of Formula I and the other pharmaceutically active agent(s) and the relative timings of administration will be selected in order to achieve the desired combined therapeutic effect. The administration in combination of a compound of Formula I salts or solvates thereof with other osteoporosis treatment agents may be in combination by administration concomitantly in: (1) a unitary pharmaceutical composition including each compound; or (2) separate pharmaceutical compositions each including one of the compounds. Alternatively, the combination may be administered separately in a sequential manner wherein one treatment agent is administered first and the other(s) subsequently or vice versa. Such sequential administration may be close in time or remote in time.

Other potential therapeutic combinations include the compounds of the present invention combined with other compounds of the present invention, growth promoting agents, growth hormone secretagogues, growth hormone releasing factor and its analogs, growth hormone and its analogs, somatomedins, alpha-ardenergic agonists, serotonin 5-HT_{D} agonists, selective serotonin reuptake inhibitors, agents that inhibit somatostatin or its release, 5-α-reductase inhibitors, aromatase inhibitors, GnRH inhibitors, parathyroid hormone, bisphosphonates, estrogen, testosterone, SERMs, progesterone receptor agonists, and/or with other modulators of nuclear hormone receptors.

In the context of treatment for the various diseases mentioned above, the compounds of the present invention may also be combined with additional therapeutic agents selected for the treatment of other symptoms or conditions which may accompany or exist together with the conditions or diseases, the treatment of which is the subject of the present invention. For example, the compounds of the present invention may be used in combination with anti-diabetic agents, anti-osteoporosis agents, anti-obesity agents, anti-inflammatory agents, anti-anxiety agents, anti-depressants, anti-hypertensive agents, anti-platelet agents, anti-thrombotic and thrombolytic agents, cardiac glycosides, cholesterol or lipid lowering agents, mineralocorticoid receptor antagonists, phosphodiesterase inhibitors, kinase inhibitors, thyroid mimetics, anabolic agents, viral therapies, cognitive disorder therapies, sleeping disorder therapies, sexual dysfunction therapies, contraceptives, cytotoxic agents, radiation therapy, anti-proliferative agents, and anti-tumor agents. Additionally, the compounds of the present invention may be combined with nutritional supplements such as amino acids, triglycerides, vitamins, minerals, creatine, piloic acid, carnitine, or coenzyme Q10.

The compounds of this invention may be made by a variety of processes, including well-known standard synthetic methods. Illustrative general synthetic methods are set out below and then specific compounds of the invention are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of Formula I.

Those skilled in the art will recognize if a chiral center exists in compounds of Formula I. Accordingly, the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

### Experimental Section

### Abbreviations:

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| Hz (Hertz); | MHz (megahertz); |
| mol (moles); | mmol (millimoles); |
| RT (room temperature); | h (hours); |
| d (days); | EI (electron impact); |
| min (minutes); | TLC (thin layer chromatography); |
| mp (melting point); | RP (reverse phase); |
| Tᵣ (retention time); | TFA (trifluoroacetic acid); |
| TEA (triethylamine); | THF (tetrahydrofuran); |
| TFAA (trifluoroacetic anhydride); | CD₃OD (deuterated methanol); |
| CDCl₃ (deuterated chloroform); | DMSO (dimethylsulfoxide); |
| SiO₂ (silica); | atm (atmosphere); |
| EtOAc (EtOAc); | CHCl₃ (chloroform); |
| HCl (hydrochloric acid); | Ac (acetyl); |
| DMF (N,N-dimethylformamide); | Me (methyl); |
| Cs₂CO₃ (cesium carbonate); | EtOH (ethanol); |
| Et (ethyl); | tBu (tert-butyl); |
| MeOH (methanol); | CH₂Cl₂ (dichloromethane); |
| MgSO₄ (magnesium sulfate); | CH₃CN (acetonitrile); |
| K₂CO₃ (potassium carbonate); | TiCl₄ (titanium tetrachloride); |
| EtOAc (EtOAc); | CO₂ (carbon dioxide); |
| Pd(OAc)₂ (palladium acetate); | Et₂O (diethyl ether); |
| P(o-tolyl)₃ (tri-o-tolylphosphine); | Na₂SO₄ (sodium sulfate); |
| NaH (sodium hydride); | DME (1,2-dimethoxyethane); |
| NaI (sodium iodide); | NaOH (sodium hydroxide); |
| NH₄Cl (ammonium chloride); | NaHCO₃ (sodium bicarbonate); |
| AlCl₃ (aluminum chloride); | (C₂H₅O)₂P(O)H (diethyl phosphite); |
| NaN₃ (sodium azide); | CBr₄ (carbon tetrabromide); |
| PPh₃ (triphenylphosphine); | CuI (copper (I) iodide); |

Pd(Ph₃P)₄ (tetrakis(triphenylphosphine)palladium (0));

| | |
|---|---|
| CuCN (copper cyanide); | (iPrO)₃B (triisopropyl borate); |
| nBuLi (butyllithium); | Na₂CO₃ (sodium carbonate); |

DMAP (4-(dimethylamino)pyridine);
eq (equivalents);
HRMS (high resolution mass spectrometry);
LCMS (liquid chromatography mass spectrometry);
LRMS (low resolution mass spectrometry);
APCI (Atmospheric Pressure Chemical Ionization);
LiHMDS (lithium bis(trimethylsilyl)amide);
Pd(Ph₃P)₂Cl₂ (dichlorobis(triphenylphosphine)palladium(II));
EDC (N-(3-dimethylaminopropyl)-N-ethyl-carbodimide;
dpppe (1,5-bis(diphenylphosphanyl)pentane;
DMAc (N,N-dimethylacetamide);
HPLC (high performance liquid chromatography);
tmeda (N,N,N,N′,-tetramethylethylenediamine);
Pd₂(dba)₃ (dipalladiumtris(dibenzylidene acetone)).

Unless otherwise noted, reagents and solvents were obtained from commercial suppliers and were used without further purification. Unless otherwise indicated, all reactions were conducted at room temperature and all temperatures are expressed in ° C. (degrees Centigrade).

Thin-layer chromatography (TLC) was performed on silica gel 60 F₂₅₄ precoated plates. Detection was effected by exposure to UV light (254 nm). Flash and flush column chromatography was performed using Silica Gel 60. Reverse phase preparative and analytical HPLC were performed using C18 columns and acetonitrile:water gradients with 0.05% TFA as a modifier.

Compound purity and characterization were determined by ¹H-NMR, liquid chromatography-mass spectrometry (LCMS), high resolution mass spectrometry (HRMS), combustion (elemental) analysis, HPLC, and melting point. Compounds of general formula I were typically found to have purities of >90%.

¹H NMR spectra were recorded on Varian INOVA-300 and Varian INOVA-400 instruments. Chemical shifts are expressed in parts per million (ppm, δ units). Coupling constants are in units of hertz (Hz). Splitting patterns describe apparent multiplicities and are designated as s (singlet), d (doublet), dd (doublet of doublet), t (triplet), q (quartet), m (multiplet), or br (broad).

Low resolution mass spectra were obtained on Micromass ZQ, Micromass ZMD, Micromass QuattroMicro, and Micromass GCT instruments from Micromass Ltd., Altricham, UK, using either Atmospheric Pressure Chemical Ionization (APCI) or ESI Ionization (ESI).

High resolution mass spectral data (HRMS) were recorded with Micromass LCT and Micromass GCT instruments.

Combustion analyses were performed by Atlantic Microlab, Inc. (Norcross, Ga.).

Melting points were recorded in open capillary tubes and are uncorrected.

The bolded numerals reference the compounds as depicted in the following schemes. For the following schemes, depending on subsequent chemistry and functional group compatibility, the phenol groups of specific intermediates may need to be protected using synthetic methods appreciated by those skilled in the art.

### Synthetic Schemes

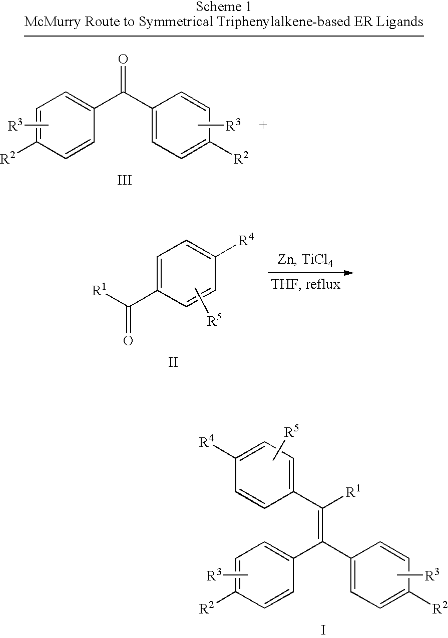

The symmetrical triphenylalkene compound I can be prepared following the route illustrated in Scheme 1. McMurry coupling between substituted benzophenone III and substituted phenyl alkyl ketone II provides the triphenylalkene I. For McMurry reaction conditions, see Mukaiyama et al., Chem. Lett. (1973), 1041; Lenoir, Synthesis, (1977), 553; Lenoir and Burghard, J. Chem. Res. (S) (1980), 396; McMurry, Chem. Rev. (1989), 89, 1513-1524; McMurry, Acc. Chem. Res. (1983) 16, 405-511; and S. Gauthier et al., J. Org. Chem., (1996), 61, 389>3893, each herein incorporated by reference with regard to such teaching.

Ketones II and III are either commercially available or may be prepared by synthetic methods appreciated by those skilled in the art (Scheme 2 and 3, for example).

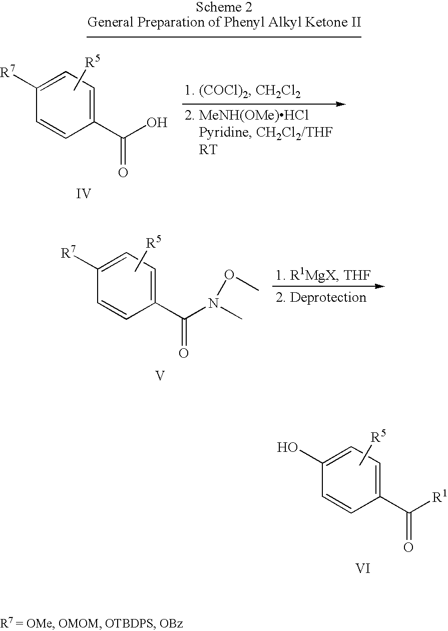

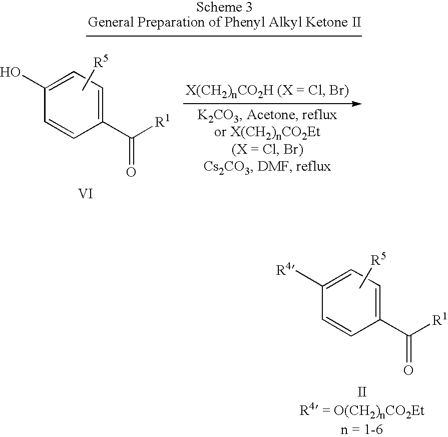

Conversion of acid IV to the acid chloride followed by treatment with N,O-dimethyl hydroxylamine hydrochloride yields the Weinreb amide V. The acid chloride can be prepared using well documented procedures familiar to those skilled in the art. Treatment of amide V with a Grignard reagent followed by demethylation/deprotection affords compound VI. For general reaction conditions, see S. Nahm and S. M. Weinreb Tetrahedron Lett. (1981), 22, 3815. B. M. Kim, et al., Tetrahedron Lett. (1994), 35, 5153, for review, see M. P. Sibi, Org. prep. Proc. Intl. (1993), 25, 15, each herein incorporated by reference with regard to such teaching.

Triphenylalkene I can also be prepared using the procedure illustrated in Scheme 4. Intermediate 1,1-dibromo-1-alkene VII can be prepared from alkyl phenyl ketone II using the procedure reported by Corey and Fuchs (see E. J. Corey and P. L. Fuchs, Tetrahedron Lett. (1972), 3769, herein incorporated by reference) as shown in Scheme 4. Alternatively, the dibromo compound VII can also be prepared using the procedure reported by V. G. Nenajdenko, et al J. Chem. Soc., Perkin Trans. I, (2002), 883, J. F. Normant et al Synthesis (2000), 109, herein incorporated by reference. Dibromo alkene VII can be coupled with a variety of aryl boronic acids VIII using Suzuki reaction conditions to afford triphenylalkene 1. For general Suzuki coupling reaction conditions, see, Miyaura, N., Suzuki, A. Chem. Rev. (1995), 95, 2457-2483; Suzuki, A., J. Organometallic Chem. (1999), 576, 147-168; and Suzuki, A. in Metal-catalyzed Cross-coupling Reactions, Diederich, F., and Stang, P. J., Eds.; Wiley-VCH: New York, (1998), pp. 49-97, each herein incorporated by reference with regard to such teaching. For Suzuki coupling reaction conditions of 1,1-dibromo-1-alkene, see M. W. Miller et al., Synleff (2001), 254, herein incorporated by reference with regard to such teaching. The dibromo alkene VII can also be transformed to 1,1-diboryl-1-alkene intermediate, which upon reaction with aryl halides can generate 1,1,2-triaryl-alkenes 1. For related transformations, see M. Shimizu et al., J. Am. Chem. Soc., (2005), 127, 12506, herein incorporated by reference with regard to such teaching.

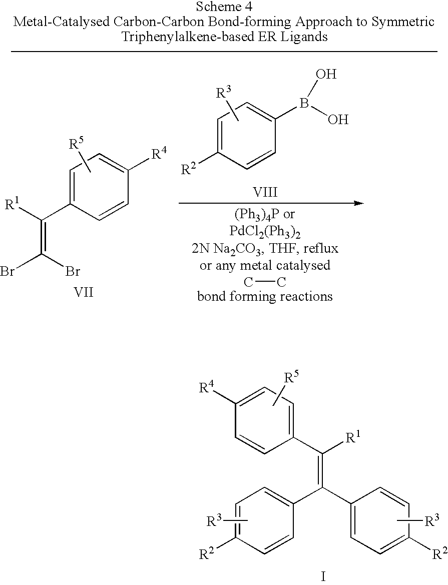

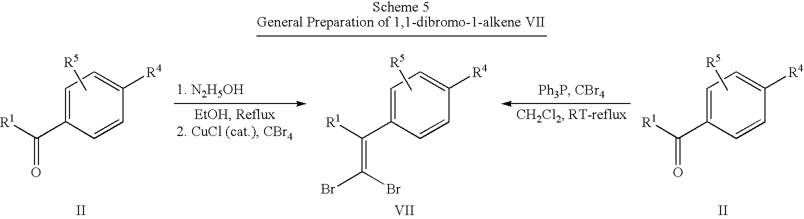

An O-alkylation of IX (Scheme 6) with alkyl halides provides compounds X. Compound X and XI can be further subjected to prepare additional analogues. For example, when the depicted R group is Et, hydrolysis of X gives XI, the corresponding alkanoic acid.

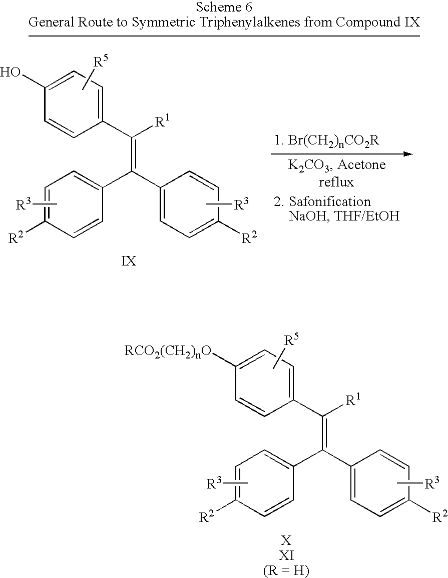

A variety of substituted symmetrical triphenylalkenes can be prepared by using the versatile intermediate XIII (Scheme 7). Compound XIII can be prepared from XII by following the literature procedure described in the art. For reaction conditions, M. Kodomari et al Tetrahedron Lett. (2001), 3105-3107, herein incorporated by reference with regard to such teaching. Transition-metal catalyzed cross coupling carbon-carbon bond forming reactions described in the art can be employed to make compound I from XII.

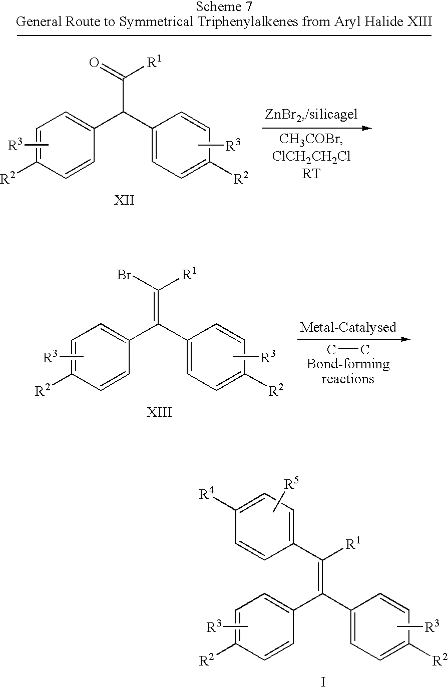

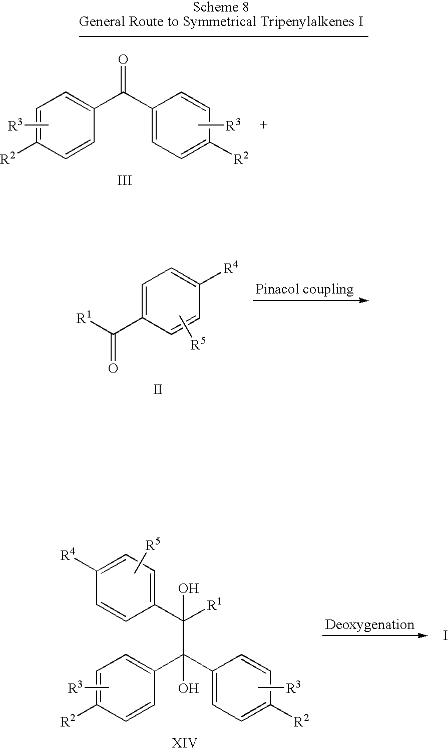

The triarylated compound I can also be prepared using the two step sequence as shown in Scheme 8. Ketones II and III can be coupled using pinacol coupling method to give the vicinal diol XIV. The diol compound XIV can be transformed to olefin I using the deoxygenation conditions that are well documented in the art. For Pinacol coupling reactions, see T. Wirth et al Angew. Chem. Int. Ed. Engl. (1996), 35, 61, X. Xu et al J. Org. Chem. (2005), 70, 8594 and leading references cited therein and for olefin synthesis by deoxygenation conditions, see E. Block in Organic Reactions (1984), 30, 457, herein incorporated by references with regard to such teaching.

### EXAMPLES

The following specific examples are included as illustrations and are not to be construed as limiting the scope of the present invention.

### Example 1 (3)

### ({4-[1-Butyl-2,2-bis(4-hydroxyphenyl)ethenyl]phenyl}oxy)acetic acid (3)

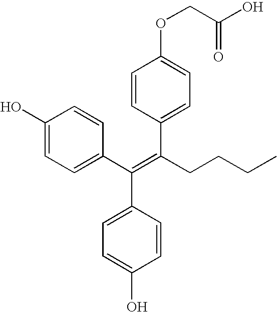

### Step 1: Ethyl[(4-pentanoylphenyl)oxy]acetate (1)

A round-bottom flask was charged with 1-(4-hydroxyphenyl)-1-pentanone (5.34 g, 30.0 mmol), ethyl bromoacetate (8.3 mL, 75.0 mmol), K₂CO₃ (8.3 g, 60 mmol), and acetone (200 mL) under N₂. The reaction mixture was refluxed for 4 h. After cooling to room temperature the reaction mixture was filtered and the filtrate concentrated under reduced pressure to give the crude product. The crude product was purified by flash SiO₂ column chromatography with hexanes: EtOAc (19:1 to 4:1) to yield 7.90 g (˜100%) of the title compound 1 as a white solid. ¹H NMR (300 MHz, CDCl₃): δ 0.96 (t, J=7.6 Hz, 3H), 1.32 (t, J=7.6 Hz, 3H), 1.43 (app. sextet, J=7.6 Hz, 1H), 1.72 (app. quintet, J=7.6 Hz, 1H), 2.93 (t, J=7.6 Hz, 2H), 4.30 (q, J=7.0 Hz, 2H), 4.69 (s, 2H), 6.95 (d, J=8.7 Hz, 2H), 7.96 (d, J=8.7 Hz, 2H). LCMS (ESI): m/z 265 (M+H)⁺.

### Step 2: Ethyl ({4-[1-butyl-2,2-bis(4-hydroxyphenyl)ethenyl]phenyl}oxy)acetate (2)

To a stirred suspension of zinc powder (3.3 g, 50 mmol) in THF (100 mL) at room temperature under nitrogen atmosphere was slowly (drop-wise) added TiCl₄ (2.7 mL, 25 mmol). The resulting reaction mixture was heated at reflux for 1 h. A mixture of bis(4-hydroxyphenyl)methanone (1.07 g, 5.0 mmol) and ketone 1 (4.0 g, 15.13 mmol) in THF (mL) was then added followed by refluxing an additional 2 h. The reaction mixture was allowed to cool at room temperature and poured into a 10% aqueous K₂CO₃ (300 mL) slowly. The reaction mixture was filtered through celite and the solids washed with EtOAc. The filtrate was extracted with EtOAc (4×150 mL). The combined organics were washed with brine, dried, filtered, and the filtrate concentrated under reduced pressure to give the crude product. The crude product was purified by flash chromatography over SiO₂ with hexanes: ethyl acetate (100:0 to 1:1) to afford 1.60 g (72%) of the title compound 2 as an off-white foam. ¹H NMR (300 MHz, DMSO-d₆): δ 0.73 (t, J=7.2 Hz, 3H), 1.89 (m, 7H), 2.33 (br t, J=7.6 Hz, 2H), 4.15 (q, J=7.2 Hz, 2H), 4.70 (s, 2H), 6.41 (d, J=8.8 Hz, 2H), 6.60 ((d, J=8.4 Hz, 2H), 6.71 (d, J=3.0 Hz, 2H), 6.74 (d, J=2.4 Hz, 2H), 6.94 (d, J=8.4 Hz, 2H), 6.99 (d, J=8.4 Hz, 2H), 9.13 (s, 1H), 9.36 (s, 1H). LCMS (ESI): m/z 445 (M−H)⁻.

### Step 3: ({4-[1-Butyl-2,2-bis(4-hydroxyphenyl)ethenyl]phenyl}oxy)acetic acid (3)

To a solution of ethyl ({4-[1-butyl-2,2-bis(4-hydroxyphenyl)ethenyl]phenyl}oxy)acetate 2 (0.218 g, 0.488 mmol) and THF/EtOH (1:1, 4 mL) was added 1 N aqueous NaOH (2 mL). The reaction mixture was stirred at 70° C. for 1 h. The reaction mixture was cooled to RT, poured into 20% aqueous HCl (50 mL), and then the mixture was extracted with EtOAc (3×30 mL). The combined organic layer was washed with brine (1×15 mL), dried over Na₂SO₄, and filtered. The filtrate was concentrated under reduced pressure to afford the crude product. The crude product was purified by flash SiO₂ column chromatography using CHCl₃: MeOH (19:1 to 4:1) to afford 150 mg (74%) of the title compound 3 as an off-white foam. ¹H NMR (300 MHz, DMSO-d₆): δ 0.73 (t, J=7.0 Hz, 3H), 1.18 (br m, 4H), 2.32 (br s, 2H), 4.32 (s, 2H), 6.41 (d, J=8.4 Hz, 2H), 6.60 (d, J=8.4 Hz, 2H), 6.65 (d, J=8.4 Hz, 2H), 6.72 (d, J=8.4 Hz, 2H), 6.93 (d, J=6.0 Hz, 2H), 6.96 (d, J=7.8 Hz, 2H). LCMS (APCI): m/z 417 (M−H)⁻.

### Example 2 (6)

### ({4-[2,2-Bis(4-hydroxyphenyl)-1-propylethenyl]phenyl}oxy)acetic acid

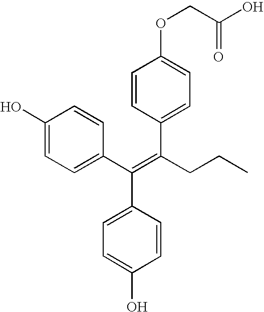

### Step 1: Ethyl[(4-butanoylphenyl)oxy]acetate (4)

The general O-alkylation procedure described for 1 (Example 1, Step 1) was employed using 1-(4-hydroxyphenyl)-1-butanone (5.0 g, 30.5 mmol) and ethyl bromoacetate (8.7 g, 61.0 mmol). Standard work-up followed by purification gave 7.6 g (99%) of the title compound 4 as a white solid. ¹H NMR (300 MHz, CDCl₃)): δ 1.00 (t, J=7.2 Hz, 3H), 1.32 (t, J=7.0 Hz, 3H), 1.77 (app. sextet, J=7.6 Hz, 2H), 2.91 (t, J=7.2 Hz, 2H), 6.95 (d, J=8.8 Hz, 2H), 7.96 (d, J=9.0 Hz, 2H). LCMS (ESI): m/z 251 (M+H)⁺.

### Step 2: Ethyl ({4-[2,2-bis(4-hydroxyphenyl)-1-propylethenyl]phenyl}oxy)acetate (5)

The general McMurry coupling procedure described for 2 (Example 1, step 2) was employed with bis(4-hydroxyphenyl)methanone (1.43 g, 6.67 mmol) and ester 4 (5.0 g, 20.0 mmol). The standard work-up followed by purification gave 2.56 g (89%) of the title compound 5 as an off-white foam. ¹H NMR (300 MHz, DMSO-d₆): δ 0.75 (t, J=7.6 Hz, 3H), 1.26-1.15 (m, 5H), 2.32 (t, J=7.2 Hz, 2H), 4.15 (q, J=7.0 Hz, 2H), 6.41 (d, J=8.4 Hz, 2H), 6.60 (d, J=8.8 Hz, 2H), 6.71 (d, J=4.2 Hz, 2H), 6.74 (d, J=4.0 Hz, 2H), 6.95 (d, J=8.4 Hz, 2H), 6.99 (d, J=8.8 Hz, 2H). LCMS (ESI): m/z 431 (M−H)⁻.

### Step 3: ({4-[2,2-Bis(4-hydroxyphenyl)-1-propylethenyl]phenyl}oxy)acetic acid (6)

The general saponification procedure described for 3 (Example 1, Step 3) was employed using ethyl ester 5 (0.325 g, 0.751 mmol) with 1 N NaOH (11.5 mL, 11.27 mmol) in 1:1 THF:EtOH (20 mL). Standard acid work-up followed by purification gave 0.240 g (79%) of the title compound 6 as an off-white foam. ¹H NMR (400 MHz, Acetone-d₆): δ 0.76 (t, J=5.4 Hz, 3H), 1.28 (br s, 2H), 2.38 (br s, 2H), 2.85 (br s, 2H), 4.56 (br s, 2H), 6.52 (d, J=8.0 Hz, 2H), 6.74 (app, t, J=8.0 Hz, 4H), 6.85 (d, J=8.4 Hz, 2H). LCMS (APCI): m/z 403 (M−H)⁻.

### Example 3 (9)

### [(4-{1-Ethyl-2,2-bis[4-(methyloxy)phenyl]ethenyl}phenyl)oxy]acetic acid (9)

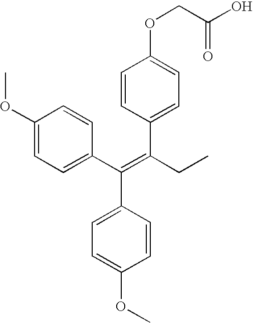

### Step 1: 4-{1-Ethyl-2,2-bis[4-(methyloxy)phenyl]ethenyl}phenol (7)

The general McMurry coupling procedure described for 2 (Example 1, step 2) was employed using bis[4-(methyloxy)phenyl]methanone (4.84 g, 20.0 mmol) and 1-(4-hydroxyphenyl)-1-propanone (9.0 g, 60.0 mmol). Standard work-up followed by purification gave 3.60 g (50%) of the title product 7 as a white solid. ¹H NMR (400 MHz, CDCl₃): δ 0.92 (t, J=7.6 Hz, 3H), 2.44 (q, J=7.2 Hz, 2H), 3.69 (s, 3H), 3.82 (s, 3H), 6.56 (d, J=8.4 Hz, 2H), 6.63 (d, J=8.4 Hz, 2H), 6.77 (d, J=8.4 Hz, 2H), 6.87 (d, J=8.8 Hz, 2H), 6.97 (d, J=8.8 Hz, 2H), 7.13 (d, J=8.4 Hz, 2H). LCMS (ESI): m/z 361 (M+H)⁺.

### Step 2: Ethyl[(4-{1-ethyl-2,2-bis[4-(methyloxy)phenyl]ethenyl}phenyl)oxy]acetate (8)

The O-alkylation procedure described for compound 1 (Example 1, Step 1) was employed using 4-{1-ethyl-2,2-bis[4-(methyloxy)phenyl]ethenyl}phenol 7 (0.850 g, 2.36 mmol), ethyl bromoacetate (0.326 mL, 2.95 mmol), K₂CO₃ (0.490 g, 3.54 mmol), and acetone (50 mL). Standard work-up followed by purification gave 0.880 g (83%) of the title compound 8 as a white solid. ¹H NMR (300 MHz, CDCl₃): δ 0.94 (t, J=7.6 Hz, 3H), 1.30 (t, J=7.2 Hz, 3H), 2.47 (q, J=7.6 Hz, 2H), 3.71 (s, 3H), 3.84 (s, 3H), 4.28 (q, J=7.2H, 2H), 4.58 (s, 2H), 6.57 (d, J=8.8 Hz, 2H), 6.74 (d, J=8.7 Hz, 2H), 6.78 (d, J=8.8 Hz, 2H), 6.89 (d, J=8.8 Hz, 2H), 7.05 (d, J=8.8 Hz, 2H), 7.15 (d, J=8.8 Hz, 2H). LCMS (ESI): m/z 469 (M+Na)⁺.

### Step 3: [(4-{1-Ethyl-2,2-bis[4-(methyloxy)phenyl]ethenyl}phenyl)oxy]acetic acid (9)

The general hydrolysis conditions described for 3 (Example 1, Step 3) was employed using ethyl [(4-{1-ethyl-2,2-bis[4-(methyloxy)phenyl]ethenyl}phenyl)oxy]acetate 8 (0.200 g, 0.45 mmol) and 1 N aqueous NaOH (5 mL) in 1:1 THF:EtOH (10 mL). Regular acid work-up followed by purification gave 0.115 g (62%) of the title compound 9 as a white solid. ¹H NMR (300 MHz, CDCl₃): δ 0.85 (t, J=7.2 Hz, 3H), 2.37 (q, J=6.6 Hz, 2H), 3.63 (s, 3H), 3.76 (s, 3H), 4.60 (s, 2H), 6.61 (d, J=7.8 Hz, 2H), 6.72 (d, J=7.8 Hz, 4H), 6.92 (d, J=8.0 Hz, 2H), 7.02 (d, J=8.0 Hz, 2H), 7.08 (d, J=7.8 Hz, 2H). LCMS (ESI): m/z 441 (M+Na)⁺ and 417 (M−H)⁻.

### Example 4 (12)

### {[4-[1-Ethyl-2,2-bis(4-hydroxyphenyl)ethenyl]-2-(methyloxy)phenyl]oxy}acetic acid (12)

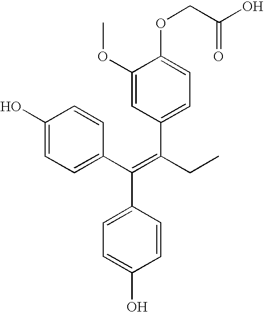

### Step 1: Ethyl {[4-butanoyl-2-(methyloxy)}acetate (10)

The general O-alkylation procedure described for 1 (Example 1, Step 1) was employed with 1-[4-hydroxy-3-(methyloxy)phenyl]-1-propanone (2.60 g, 14.44 mmol) and ethyl bromoacetate (6.0 mL, 43.33 mmol). Standard work-up followed by purification gave 3.710 g (96%) of the title compound 10 as a white solid. ¹H NMR (400 MHz, CDCl₃): δ 1.20 (t, J=7.6 Hz, 3H), 1.27 (t, J=6.8 Hz, 3H), 2.94 (q, J=7.2 Hz, 2H), 4.25 (q, J=7.2 Hz, 2H), 4.74 (s, 2H), 6.87 (d, J=8.4 Hz, 1H), 7.52 (d, J=2.0 Hz, 1H), 7.55 and 7.53 (dd, J₁=8.4 Hz, J₂=2.0 Hz, 1H).

### Step 2: Ethyl {[4-[1-ethyl-2,2-bis(4-hydroxyphenyl)ethenyl]-2-(methyloxy)phenyl]oxy}acetate (11)

The general McMurry coupling procedure described for 2 (Example 2, step 2) was employed using bis(4-hydroxyphenyl)methanone (0.885 g, 4.13 mmol), ethyl {[4-butanoyl-2-(methyloxy)phenyl]oxy}acetate 11 (3.30 g, 12.4 mmol), TiCl₄.2THF complex (8.276 g, 24.78 mmol) (note: in this experiment neat TiCl₄ was substituted with solid TiCl₄.2THF). Standard work-up followed by purification gave 1.668 g (90%) of the title compound 11 as an off-white solid. ¹H NMR (400 MHz, DMSO-d₆): δ 0.84 (t, J=7.6 Hz, 3H), 1.16 (t, J=7.2 Hz, 3H), 2.37 (q, J=7.6 Hz, 2H), 3.49 (s, 3H), 4.12 (q, J=7.2 Hz, 2H), 4.65 (s, 2H), 6.40 (d, J=8.8 Hz, 2H), 6.60-6.58 (m, 4H), 6.65 (d, J=8.8 Hz, 1H), 6.71 (d, J=8.4 Hz, 2H), 6.93 (d, J=8.4 Hz, 2H), 9.14 (s, 1H), 9.36 (s, 1H). LCMS (APCI): m/z 471. (M+Na)⁺ and 449 (M+H)⁺.

### Step 3: {[4-[1-Ethyl-2,2-bis(4-hydroxyphenyl)ethenyl]-2-(methyloxy)phenyl]oxy}acetic acid (12)

The general saponification procedure described for 3 (Example 1, Step 3) was employed using ethyl ester 11 (0.140 g, 0.312 mmol) and 1 N NaOH (2 mL) in 1:1 THF:EtOH (6 mL). Standard acid work-up followed by purification gave 0.110 g (84%) of the title compound 12 as an off-white solid. mp 253-254° C. ¹H NMR (400 MHz, CD₃OD): δ 0.92 (t, J=7.6 Hz, 3H), 2.48 (q, J=7.6 Hz, 2H), 3.52 (s, 3H), 4.32 (s, 2H), 6.42 (d, J=8.4 Hz, 2H), 6.62 (br s, 1H), 6.67 (d, J=8.4 Hz, 1H), 6.70 (d, J=8.8 Hz, 2H), 6.75 (d, J=8.4 Hz, 3H), 7.00 (d, J=8.4 Hz, 2H). LCMS (APCI): m/z 419. (M−H)⁻ and 421.03 (M+H)⁺.

### PROPHETIC EXAMPLES

The following examples may be prepared by methods analogous to those herein described:

### Example 5 (#)

### {[4-[(2,2-Bis(4-hydroxyphenyl)-1-propylethenyl]-2-(methyloxy)phenyl]oxy}acetic acid

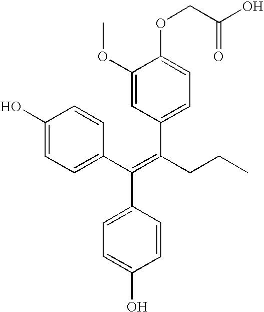

### Example 6 (#)

### {[4-[1-Butyl-2,2-bis(4-hydroxyphenyl)ethenyl]-2-(methyloxy)phenyl]oxy}acetic acid

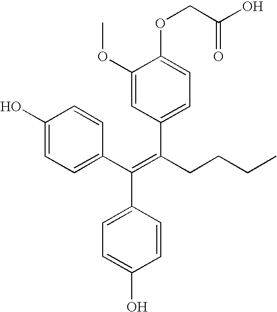

### Example 7 (#)

### {[4-[1-Ethyl-2,2-bis(4-hydroxyphenyl)ethenyl]-2-(trifluoromethyl)phenyl]oxy}acetic acid

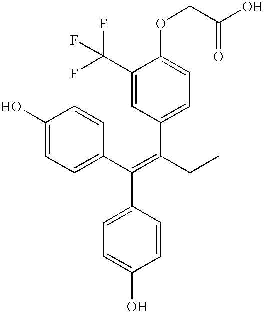

### Example 8 (#)

### {[4-[2,2-Bis(4-hydroxyphenyl)-1-propylethenyl]-2-(trifluoromethyl)phenyl]oxy}acetic acid

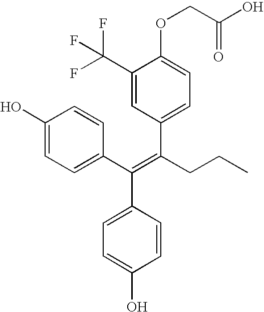

### Example 9 (#)

### {[4-[1-Butyl-2,2-bis(4-hydroxyphenyl)ethenyl]-2-(trifluoromethyl)phenyl]oxy}acetic acid

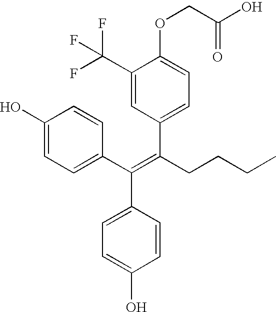

### Example 10 (#)

### ({4-[1-Ethyl-2,2-bis(4-hydroxyphenyl)ethenyl]-2-fluorophenyl}oxy)acetic acid

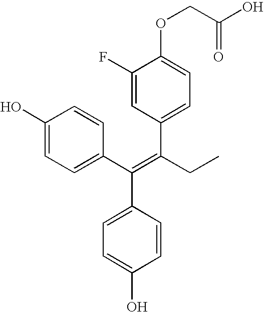

### Example 11 (#)

### ({4-[2,2-Bis(4-hydroxyphenyl)-1-propylethenyl]-2-fluorophenyl}oxy)acetic acid

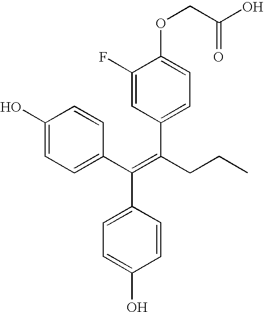

### Example 12 (#)

### ({4-[1-Butyl-2,2-bis(4-hydroxyphenyl)ethenyl]-2-fluorophenyl}oxy)acetic acid

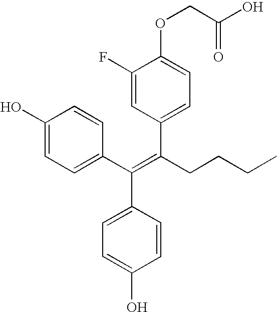

### Example 13 (#

### 2-({4-[1-Butyl-2,2-bis(4-hydroxyphenyl)ethenyl]phenyl}oxy)-2-methylpropanoic Acid

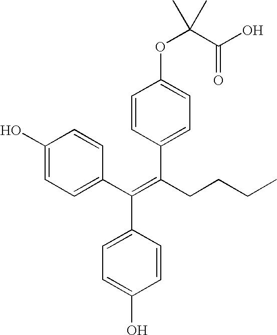

### Example 14

### ({4-[2,2-bis(4-hydroxyphenyl)-1-pentylethenyl]phenyl}oxy)acetic acid

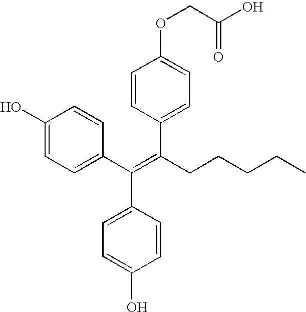

### Example 15

### ({4-[2,2-bis(4-hydroxyphenyl)-1-pentylethenyl]-2-fluorophenyl}oxy)acetic Acid

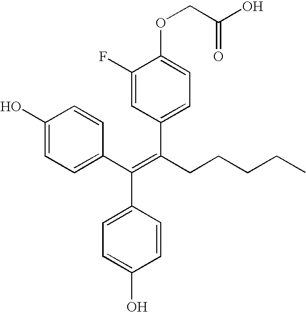

### Biological Data

ER alpha Fluorescence polarisation assays.

Assays were conducted using both full length and ligand binding domain protein

Full length ER alpha—The assay was performed using a commercially available kit (P3029, Invitrogen, Carlsbad, Calif.). The assay was performed according to the manufacturer's protocol with minor amendments Namely, 15 nM ERα and 1 mM Fluormone EL Red were dissolved and mixed in Complete ER Red Buffer. 10 μl of the mix was dispensed to each well of Greiner low volume plates—Black solid low volume 384-well plates—(Greiner-Product No. 784076), containing compounds within the concentration range of 10⁻⁵-10⁻¹²M in dimethyl sulfoxide (DMSO). The plates were spun for 1 min at 200 g, covered to protect the reagents from light, and then incubated at room temperature for 2 hours. Plates were read on an Acquest, LJL Biosystems, Sunnyvale, Calif., using a 530-25 nm excitation and 580-10 nm emission interference filter and a 561 nm Dichroic mirror.

### Expression and Purification of ERα LBD

A cDNA sequence corresponding to residues 297 to 555 of human ERα (accession number NP_{—}000116.2) was cloned into a pET24vector (Novagen, San Diego, Calif.) with a N-terminal hexa-histidine tag. The plasmid was transformed into E. Coli BL21-DE3 cells. Cells were grown at 23° C. for 18 hr, the temperature was lowered to 18° C. before addition of 250 μM IPTG. Cells were grown an additional 24 hr before harvesting. Cells were lysed in 50 mM TRIS pH 8.0/250 mM NaCl/2 M Urea and spun down. The supernatant was made 50 mM in imidazole and loaded onto a Ni-chelating sepharose column (Pharmicia) and eluted with a linear gradient of 50 to 500 mM Imidazole. Fractions containing ERα LBD were pooled and dialyzed against 50 mM TRIS pH 8.0/250 mM NaCl/5 mM DTT and 10% glycerol. Samples were aliquoted and frozen at −70° C.

The assay was performed by mixing 15 nM ER alpha LBD with 1 nM Fluormone-EL-Red (Invitrogen No. P3030) in assay buffer (Tris-HCl (50 mM; pH8), KCl, (500 mM), Dithiothreitol (1 mM), Ethylene diamine tetraacetic acid (1 mM), glycerol (10% v/v), 3 cholamidopropyl-dimethylammonio1-propanesulfonate-(2 mM), Sodium orthovanadate (1 mM—this was prepared as 100 mM stock by dissolving in distilled water and 2 successive rounds of adjusting pH to 10, boiling and cooling)). 101 of the mix was dispensed to each well of Greiner low volume plates—Black solid low volume 384-well plates—(Greiner, Longwood, Fla.—Product No. 784076), containing compounds within the concentration range of 10⁻⁵-10⁻¹²M in dimethyl sulfoxide (DMSO). The plates were spun for 1 min at 200 g, covered to protect the reagents from light, and then incubated at room temperature for 2 hours. Plates were read on the Acquest using a 530-25 nm excitation and 580-10 nm emission interference filter and a 561 nm Dichroic mirror.

### ER Beta Fluorescence Polarisation Assays.

Assays were conducted using both full length and ligand binding domain protein. Full length ER beta—The assay was performed using a commercially available kit (P3032, Invitrogen). The assay was performed according to the manufacturer's protocol with minor amendments. Namely, 30 nM ERβ and 1 nM Fluormone EL Red were dissolved and mixed in Complete ER Red Buffer. 10 μg of the mix was dispensed to each well of Greiner low volume plates—Black solid low volume 384-well plates—(784076, Greiner), containing compounds within the concentration range of 10⁻⁵-10⁻¹²M in dimethyl sulfoxide (DMSO). The plates were spun for 1 min at 200 g, covered to protect the reagents from light, and then incubated at room temperature for 2 hours. Plates were read on an Acquest (Acquest/Biosystems) using a 530-25 nm excitation and 580-10 nm emission interference filter and a 561 nm Dichroic mirror.

### Expression and Purification of ERβ LBD

A cDNA sequence corresponding to residues 257 to 530 of human ERβ (accession number NP_{—}001428.1) was cloned into a pRSETa (Novagen) vector with a N-terminal hexa-histidine tag. The plasmid was transformed into E. Coli BL21-DE3 cells. The cells were grown at 23° C. for 18 hr, the temperature was lowered to 18° C. and then 250 μM of IPTG was added. Cells were grown an additional 24 hr before harvesting. Cells were lysed in 50 mM TRIS pH 8.0/250 mM NaCl and spun down. The supernatant wais made 50 mM in imidazole and loaded onto a Ni-chelating sepharose column (Amersham Pharmacia Biotech, Piscataway, N.J.) and eluted with a linear gradient of 50 to 500 mM Imidazole. Fractions containing ERβ LBD were pooled and diluted to 50 mM NaCl and loaded on a Q-sepharose column (Pharmacia) equilibrated with 50 mM TRIS pH 8.0/50 mM NaCl/5 mM DTT and 10% glycerol. The ERβ was eluted with a linear gradient from 50 mM to 500 mM NaCl. Fractions containing ERβ LBD were pooled and dialyzed against 50 mM TRIS pH 8.0/250 mM NaCl/5 mM DTT and 10% glycerol. Samples were aliquoted and frozen at −70° C.

The assay was performed by mixing 30 nM ER beta LBD with 1 nM Fluormone-EL-Red (Invitrogen No. P3030) in assay buffer (Tris-HCl (50 mM; pH8), KCl, (500 mM), Dithiothreitol (1 mM), Ethylene diamine tetraacetic acid (1 mM), glycerol (10% v/v), 3 cholamidopropyl-dimethylammonio1-propanesulfonate-(2 mM), Sodium orthovanadate (1 mM—this was prepared as 100 mM stock by dissolving in distilled water and 2 successive rounds of adjusting pH to 10, boiling and cooling)). 10 μl of the mix was dispensed to each well of black solid low volume 384-well plates—(784076, Greiner), containing compounds within the concentration range of 10⁻⁵-10⁻¹²M in dimethyl sulfoxide (DMSO). The plates were spun for 1 min at 200 g, covered to protect the reagents from light, and then incubated at room temperature for 2 hours. Plates were read on the Acquest using a 530-25 nm excitation and 580-10 nm emission interference filter and a 561 nm Dichroic mirror.

### Data Analysis

All data was normalized to the mean of 16 high and 16 low control wells on each plate. A four parameter curve fit of the following form was then applied

$$y = \frac{a - d}{1 + {( \frac{x}{c} )}^{b}} + d$$

Where a is the minimum, b is the Hill slope, c is the IC₅₀ and d is the maximum. Data is presented as the mean pIC₅₀ with the standard deviation of the mean of n experiments.

The compounds of the Examples above exhibited pIC₅₀ values ranging from 6 to 8.5.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the Invention are intended to be included with the scope of the appended claims.

What is claimed is:

1. A compound of Formula I 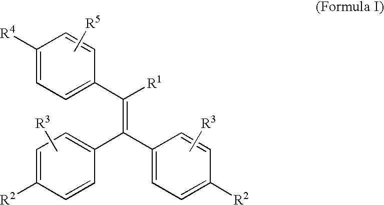
or a pharmaceutically acceptable salt or solvate thereof, wherein
each R³ is the same and selected from the group consisting of hydrogen, hydroxy, C₁-C₆ alkyl, halogen, C₁-C₆ alkoxy, and C₁-C₆ haloalkyl;
R⁴ is —OCH₂C(O)OH; and
R¹ is selected from the group consisting of C₁-C₆ alkyl and C₁-C₆ haloalkyl;
each R² is the same and selected from the group consisting of hydroxy, C₁-C₄ alkoxy, and halogen; and
R⁵ is selected from the group consisting of hydroxy, C₁-C₆ alkyl, halogen, C₁-C₆ alkoxy, or C₁-C₆ haloalkyl;
or
R¹ is selected from the group consisting of C₁-C₆ alkyl and C₁-C₆ haloalkyl;
each R² is the same and selected from the group consisting of C₁-C₄ alkoxy, and halogen; and
R⁵ is selected from the group consisting of hydrogen, hydroxy, C₁-C₆ alkyl, halogen, C₁-C₆ alkoxy, or C₁-C₆ haloalkyl;
or
R¹ is selected from the group consisting of C₃-C₆ alkyl and C₁-C₆ haloalkyl;
each R² is the same and selected from the group consisting of hydroxy, C₁-C₄ alkoxy, and halogen; and
R⁵ is selected from the group consisting of hydrogen, hydroxy, C₁-C₆ alkyl, halogen, C₁-C₆ alkoxy, or C₁-C₆ haloalkyl.

2. The compound of claim 1 or a pharmaceutically acceptable salt or solvate thereof, wherein R¹ is selected from the group consisting of C₁-C₆ alkyl and C₁-C₆ haloalkyl;
each R² is the same and selected from the group consisting of hydroxy, C₁-C₄ alkoxy, and halogen; and
R⁵ is selected from the group consisting of hydroxy, C₁-C₆ alkyl, halogen, C₁-C₆ alkoxy, or C₁-C₆ haloalkyl.

3. The compound of claim 2 or a pharmaceutically acceptable salt or solvate thereof, wherein R¹ is selected from C₂-C₆ alkyl.

4. The compound of claim 2 or a pharmaceutically acceptable salt or solvate thereof, wherein R² is hydroxy.

5. The compound of claim 1 or a pharmaceutically acceptable salt or solvate thereof, wherein R¹ is selected from the group consisting of C₁-C₆ alkyl and C₁-C₆ haloalkyl;
each R² is the same and selected from the group consisting of C₁-C₄ alkoxy, and halogen; and
R⁵ is selected from the group consisting of hydrogen, hydroxy, C₁-C₆ alkyl, halogen, C₁-C₆ alkoxy, or C₁-C₆ haloalkyl.

6. The compound of claim 5 or a pharmaceutically acceptable salt or solvate thereof, wherein R¹ is selected from C₂-C₆ alkyl.

7. The compound of claim 5 or a pharmaceutically acceptable salt or solvate thereof, wherein R⁵ is hydrogen.

8. The compound of claim 1 or a pharmaceutically acceptable salt or solvate thereof, wherein R¹ is selected from the group consisting of C₃-C₆ alkyl and C₁-C₆ haloalkyl;
each R² is the same and selected from the group consisting of hydroxy, C₁-C₄ alkoxy, and halogen; and
R⁵ is selected from the group consisting of hydrogen, hydroxy, C₁-C₆ alkyl, halogen, C₁-C₆ alkoxy, or C₁-C₆ haloalkyl.

9. The compound of claim 8 or a pharmaceutically acceptable salt or solvate thereof, wherein R¹ is selected from C₃-C₆ alkyl.

10. The compound of claim 8 or a pharmaceutically acceptable salt or solvate thereof, wherein R² is hydroxy.

11. The compound of claim 8 or a pharmaceutically acceptable salt or solvate thereof, wherein R⁵ is hydrogen.

12. The compound of claim 1 or a pharmaceutically acceptable salt or solvate thereof, wherein R⁵ is trifluoro methyl.

13. A compound selected from:
({4-[1-butyl-2,2-bis(4-hydroxyphenyl)ethenyl]phenyl}oxy)acetic acid;
({4-[2,2-bis(4-hydroxyphenyl)-1-propylethenyl]phenyl}oxy)acetic acid;
[(4-{1-ethyl-2,2-bis[4-(methyloxy)phenyl]ethenyl}phenyl)oxy]acetic acid; and
{[4-[1-ethyl-2,2-bis(4-hydroxyphenyl)ethenyl]-2-(methyloxy)phenyl]oxy}acetic acid, or a pharmaceutically acceptable salt or solvate thereof.

14. A pharmaceutical composition comprising the compound according to claim 1 or a pharmaceutically acceptable salt or solvate thereof and a pharmaceutically acceptable carrier, diluent or excipient.

15. (canceled)

16. (canceled)

17. (canceled)

18. (canceled)

19. A pharmaceutical composition comprising at the compound of claim 1 or a pharmaceutically acceptable salt or solvate thereof in combination with other therapeutic agents selected from a bone building agent, anti-bone resorption agent, growth promoting agents, growth hormone secretagogues, growth hormone releasing factor and its analogs, growth hormone and its analogs, somatomedins, alpha-ardenergic agonists, serotonin 5-HT_{D} agonists, selective serotonin reuptake inhibitors, agents that inhibit somatostatin or its release, 5-α-reductase inhibitors, aromatase inhibitors, GnRH inhibitors, parathyroid hormone, bisphosphonates, estrogen, testosterone, SERMs, progesterone receptor agonists, and other modulators of nuclear hormone receptors.

20. (canceled)

21. (canceled)

22. A method of treatment for a condition or disorder affected by selective estrogen regulator modulation in a mammal in need thereof comprising administering a therapeutically effective amount of the compound of claim 1 or a pharmaceutically acceptable salt or solvate thereof,
wherein the condition or disorder is selected from osteoporosis, bone demineralization, reduced bone mass, density, or growth, osteoarthritis, acceleration of bone fracture repair and healing, acceleration of healing in joint replacement, periodontal disease, acceleration of tooth repair or growth, Paget's disease, osteochondrodysplasias, muscle wasting, the maintenance and enhancement of muscle strength and function, frailty or age-related functional decline (“ARFD”), sarcopenia, chronic fatigue syndrome, chronic myaligia, acute fatigue syndrome, acceleration of wound healing, maintenance of sensory function, chronic liver disease, AIDS, weightlessness, burn and trauma recovery, thrombocytopenia, short bowel syndrome, irritable bowel syndrome, inflammatory bowel disease, Crohn's disease and ulcerative colitis, obesity, eating disorders including anorexia associated with cachexia or aging, hypercortisolism and Cushing's syndrome, cardiovascular disease or cardiac dysfunction, congestive heart failure, high blood pressure, breast cancer, malignant tumore cells containing the androgen receptor including breast, brain, skin, ovary, bladder, lymphatic, liver, kidney, uterine, pancreas, endometrium, lung, colon, and prostate, prostatic hyperplasia, hirsutism, acne, seborrhea, androgenic alopecia, anemia, hyperpilosity, adenomas and neoplasis of the prostate, hyperinsulinemia, insulin resistance, diabetes, syndrome X, dyslipidemia, urinary incontinence, artherosclerosis, libido enhancement, sexual dysfunction, depression, depressive symptoms, nervousness, irritability, stress, reduced mental energy and low self-esteem, improvement of cognitive function, endometriosis, polycystic ovary syndrome, counteracting preeclampsia, premenstral syndrome, contraception, uterine fibroid disease, and/or aortic smooth muscle cell proliferation, vaginal dryness, pruritis, dyspareunia, dysuria, frequent urination, urinary tract infections, hypercholesterolemia, hyperlipidemia, peripheral vascular disease, restenosis, vasospasm, vascular wall damage due to immune responses, Alzheimer's disease, bone disease, aging, inflammation, rheumatoid arthritis, respiratory disease, emphysema, reperfusion injury, viral hepatitis, tuberculosis, psoriasis, systemic lupus erythematosus, amyotrophic lateral sclerosis, stroke, CNS trauma, dementia, neurodegeneration, breast pain and dysmenorrhea, menopausal or postmenopausal disorders, vasomotor symptoms, urogenital or vulvar vaginal atrophy, atrophic vaginitis, female sexual dysfunction, for enhancing libido, for the treatment of hypoactive sexual disorder, sexual arousal disorder, for increasing the frequency and intensity of orgasms, vaginismus, osteopenia, endometriosis, BPH (benign prostatic hypertrophy), dysmenorrhea, autoimmune diseases, Hashimoto's thyroiditis, SLE (systemic lupus erythematosus), myasthenia gravis, or reperfusion damage of ischemic myocardium.

23. The method of claim 22, wherein the disorder or condition is selected from menopausal or postmenopausal disorders, vasomotor symptoms, urogenital or vulvar vaginal atrophy, atrophic vaginitis, endometriosis, female sexual dysfunction, breast cancer, depressive symptoms, diabetes, bone demineralization, and osteoporosis.
